(19) United States
(12) Patent Application Publication
(10) Pub. No.: US 2017/0246049 A1
(43) Pub. Date: Aug. 31, 2017

(54) DISPOSABLE UNDERGARMENT AND RELATED DISPOSAL METHODS

(71) Applicants: Fredrica V. Coates, Winston-Salem, NC (US); Rachel H. Star, Chicago, IL (US)

(72) Inventors: Fredrica V. Coates, Winston-Salem, NC (US); Rachel H. Star, Chicago, IL (US)

(21) Appl. No.: 15/487,270

(22) Filed: Apr. 13, 2017

Related U.S. Application Data
(63) Continuation-in-part of application No. 14/825,945, filed on Aug. 13, 2015.

(51) Int. Cl. A61F 13/49; A61F 13/515; A61F 13/62; A61F 13/496

(52) U.S. Cl. CPC A61F 13/49014; A61F 13/496; A61F 13/515; A61F 13/625; A61F 2013/55195

(57) ABSTRACT

A disposable undergarment is disclosed which includes a main body having an absorbent core, a first side and a second side. The main body has a top sheet and a liquid-impermeable back sheet formed from polypropylene. The main body also has a front portion, a rear portion, and a crotch portion, and each of the front and rear portions has a top edge. The undergarment also has first and second elastic extensions each having a proximal end and a distal end. The proximal ends of the first and second extensions are ultrasonically bonded to the main body. The undergarment further has first and second attachment areas, each having a plurality of engaging elements, secured to the liquid permeable top sheet. The engaging elements engage with the first and second elastic extensions to secure the undergarment at least about 0.5 inches below said wearer's waist.

### CROSS REFERENCE TO RELATED APPLICATION

This application is a Continuation-In-Part application and claims priority to Non-provisional application, U.S. Ser. No. 14/825,945 filed Aug. 13, 2015.

### BACKGROUND OF THE INVENTION

Disposable undergarments are often used by individuals in order to capture urinary leakage attributable to incontinence. Incontinence can be attributed to a variety of medical conditions. Individuals with such conditions may use disposable undergarments to capture involuntary urinary leakage. Such individuals require disposable undergarments that can be worn under clothing.

Known disposable undergarments suffer from many disadvantages. Known disposable undergarments are often bulky, poorly fitting, inconvenient for use in an active lifestyle and create problems during disposal. Many known disposable undergarments are of the pull-on type. As such, the disposable undergarment may require a wearer to remove his or her pants in order to remove and replace the disposable undergarment. This can be a time-consuming and cumbersome activity that does not work well for individuals with active or busy lifestyles. In addition, known disposable undergarments are often cumbersome due to the fact that they are designed to retain both solid and liquid wastes and are also designed to be one-size fits all products. Many disposable undergarments do not conform to a wearer's body and can be seen through a wearers clothing due to the attachment schemes, bulky absorbent cores and general over-engineering of the products. There exists a need, therefore, for a disposable undergarment that can be easily concealed, easily removed and replaced during normal daily life and that can accommodate a variety of body types and sizes.

Known disposable undergarments also can be difficult to dispose of easily and sanitarily after they have become soiled. It can be difficult to retain the captured wastes in the soiled undergarment once it has been removed from a wearer. There exists a need for a disposable undergarment that can retain bodily fluids and wastes and can be packaged or configured to retain such fluid and wastes from a point of removal to a point of disposal.

Now a disposable undergarment has been invented, along with a method of affixing the disposable undergarment to a human body such that it is positioned at least about 0.5 inches below the wearer's waist, and a method of removing and disposing of a soiled disposable undergarment.

### SUMMARY OF THE INVENTION

Briefly, this invention relates to a disposable undergarment to be worn on a wearer's body below their waist. The disposable undergarment includes a main body having a longitudinal central axis, an absorbent core aligned along the longitudinal central axis, and a first side and a second side. Each of the first and second sides is located laterally outboard of the absorbent core. The main body also includes a liquid permeable top sheet which contacts a wearer's body and a liquid-impermeable back sheet spaced away from the wearer's body. The main body has a front portion, a rear portion, and a crotch portion located between the front and rear portions, and each of the front and rear portions has a top edge. The disposable undergarment also includes a first elastic extension and a second elastic extension, each constructed of at least about 75% polyester, at least about 3% spandex, and at least about 15% nylon. Each of the first and second elastic extensions has a proximal end and a distal end. The first elastic extension is ultrasonically bonded, at the proximal end, to the first side, and said second elastic extension is ultrasonically bonded, at the proximal end, to the second side. Each of the first and second elastic extensions extends from the top edge of the rear portion toward the crotch portion. The disposable undergarment further includes a first attachment area and a second attachment area, each having a plurality of engaging elements. Each of the first and second attachment areas is secured to the liquid permeable top sheet and each extends longitudinally from the top edge of the front portion toward the crotch portion. The plurality of engaging elements on the first attachment area engage with the first elastic extension and the plurality of engaging elements on the second attachment area engage with the second elastic extension to secure the disposable undergarment at least about 0.5 inches below the wearer's waist.

In a second embodiment, the invention relates to a disposable undergarment to be worm on a wearer's body below their waist. The disposable undergarment includes a main body having a longitudinal central axis, an absorbent core aligned along the longitudinal central axis, and a first side and a second side. Each of the first and second sides is located laterally outboard of the absorbent core. The main body has an hourglass shape and includes a liquid permeable top sheet which contacts a wearer's body and a liquid-impermeable back sheet, formed from polypropylene, spaced away from the wearer's body. The main body has a front portion, a rear portion, and a crotch portion located between the front and rear portions, and each of the front and rear portions has a top edge. The disposable undergarment also includes a first elastic extension and a second elastic extension, each constructed of at least about 79% polyester, at least about 4% spandex and at least about 16% nylon. Each of the first and second elastic extensions has a proximal end and a distal end. The first elastic extension is ultrasonically bonded, at the proximal end, to the first side, and the second elastic extension is ultrasonically bonded, at the proximal end, to the second side. Each of the first and second elastic extensions extends from the top edge of the rear portion toward the crotch portion. The disposable undergarment further includes a first attachment area and a second attachment area, each having a plurality of engaging elements. Each of the first and second attachment areas is secured to the liquid permeable top sheet and each extends longitudinally from the top edge of the front portion toward the crotch portion. The plurality of engaging elements on the first attachment area engage with the first elastic extension and the plurality of engaging elements on the second attachment area engage with the second elastic extension to secure the disposable undergarment at least about 1 inch below the wearer's waist.

A method of affixing a disposable undergarment around a crotch portion of a wearer's body and below their waist is also disclosed. The wearer's body has a buttock and hips. The method includes the steps of utilizing a disposable undergarment which includes a main body having a longitudinal central axis, an absorbent core aligned along the longitudinal central axis, and a first side and a second side. Each of the first and second sides is located laterally outboard of the absorbent core. The main body has a liquid permeable top sheet which contacts a wearer's body and a liquid-impermeable back sheet spaced away from the wearer's body. The main body also has a front portion, a rear portion, and a crotch portion located between the front and rear portions. Each of the front and rear portions has a top edge. The disposable undergarment also includes a first elastic extension and a second elastic extension, each constructed of at least about 75% polyester, at least about 3% spandex, and at least about 15% nylon. Each of the first and second elastic extensions has a proximal end and a distal end. The first elastic extension is ultrasonically bonded, at the proximal end, to the first side, and the second elastic extension is ultrasonically bonded, at the proximal end, to the second side. Each of the first and second elastic extensions extends from the top edge of the rear portion toward the crotch portion. The disposable undergarment further includes a first attachment area and a second attachment area, each having a plurality of engaging elements. Each of the first and second attachment areas is secured to the liquid permeable top sheet and each extends longitudinally from the top edge of the front portion toward the crotch portion. The plurality of engaging elements on the first attachment area engage with the first elastic extension and the plurality of engaging elements on the second attachment area engage with the second elastic extension to secure the disposable undergarment around the wearer's body and below the waist. The method also includes holding the rear portion of the disposable undergarment against the wearer's buttock and below the wearer's waist. The front portion is then wrapped around the crotch portion of the wearer's body. The method further includes stretching the first elastic extension around one of the wearer's hips and securing the first elastic extension to the plurality of engaging elements formed on the first attachment area. Lastly, the method includes stretching the second elastic extension around the wearer's other hip and securing the second elastic extension to the plurality of engaging elements formed on the second attachment area, such that the disposable undergarment is secured at least about 0.5 inches below the wearer's waist.

A method of removing a soiled disposable undergarment from around a crotch portion of a wearer's body is also disclosed. The wearer's body having a buttock and hips. The method includes the steps of utilizing a disposable undergarment which includes a main body having a longitudinal central axis, an absorbent core aligned along the longitudinal central axis, and a first side and a second side. Each of the first and second sides is located laterally outboard of the absorbent core. The main body has a liquid permeable top sheet which contacts a wearer's body and a liquid-impermeable back sheet spaced away from the wearer's body. The main body also has a front portion, a rear portion, and a crotch portion located between the front and rear portions. Each of the front and rear portions has a top edge. The disposable undergarment also includes a first elastic extension and a second elastic extension, each constructed of at least about 75% polyester, at least about 3% spandex, and at least about 15% nylon. Each of the first and second elastic extensions has a proximal end and a distal end. The first elastic extension is ultrasonically bonded, at the proximal end, to the first side, and the second elastic extension is ultrasonically bonded, at the proximal end, to the second side. Each of the first and second elastic extensions extends from the top edge of the rear portion toward the crotch portion. The disposable undergarment further includes a first attachment area and a second attachment area, each having a plurality of engaging elements. Each of the first and second attachment areas is secured to the liquid permeable top sheet and each extends longitudinally from the top edge of the front portion toward the crotch portion. The plurality of engaging elements on the first attachment area engage with the first elastic extension, and the plurality of engaging elements on the second attachment area engage with the second elastic extension to secure the disposable undergarment at least about 0.5 inches below the wearer's waist. The method also includes releasing the first and second elastic extensions from the plurality of engaging elements formed on the first and second attachment areas. The disposable undergarment is then moved away from the wearer's body whereby the wearer's hips and buttock are no longer contacted by the disposable undergarment. The method further includes rolling or folding the front portion inward upon itself up towards the rear portion to create an approximately cylindrical configuration with the first and second elastic extensions extending outward therefrom. The first and second elastic extensions are then fastened together whereby the approximately cylindrical configuration is at least partially encased. In this configuration, the soiled disposable undergarment can be disposed of in a trash receptacle.

The general object of this invention is to provide a disposable undergarment to be worn on a wearer's body and at least about 0.5 inches below their waist. A more specific object of this invention is to provide a disposable undergarment having first and second elastic extensions, each constructed of at least about 75% polyester, at least about 3% spandex, and at least about 15% nylon.

Another object of this invention is to provide a disposable undergarment which is easy to secure to a wearer's body and to remove from a wearer's body.

Still another object of this invention is to provide a disposable undergarment which utilizes first and second elastic extensions which can be ultrasonically bonded to a liquid permeable top sheet formed of spunbond and to a liquid-impermeable back sheet formed of polypropylene.

Still further, an object of this invention is to provide a method of affixing the disposable undergarment to a wearer's body.

Still further, an object of this invention is to provide a method of removing and disposing of a soiled disposable undergarment.

Other objects and advantages of the present invention will become more apparent to those skilled in the art in view of the following description and the accompanying drawings.

### BRIEF DESCRIPTION OF THE DRAWINGS

FIG. 1 is a plane view of a disposable undergarment showing the liquid permeable top sheet.

FIG. 2 is a plane view of the disposable undergarment shown in FIG. 1 showing the liquid-impermeable back sheet.

FIG. 3 is a side view of the disposable undergarment shown in FIG. 1 when positioned on a human body and aligned below the wearer's waist by at least about 0.5 inches.

FIG. 4 is a front view of a person wearing the disposable undergarment shown in FIG. 1.

FIG. 5 is a plane view of a second embodiment of a disposable undergarment showing the liquid permeable top sheet.

FIG. 6 is a plane view of the disposable undergarment shown in FIG. 5 showing the liquid-impermeable back sheet.

FIG. 7 is a plane view of the disposable undergarment shown in FIG. 1 with the front portion being partially rolled or folded over itself and towards the rear portion when in the process of being prepared for disposal.

FIG. 8 is a schematic view of the disposable undergarment when in a fastened position and ready for disposal into a trash receptacle.

FIG. 9 is a plane view of a third embodiment of a disposable undergarment showing the liquid permeable top sheet.

FIG. 10 is a plane view of the disposable undergarment shown in FIG. 9 showing the liquid-impermeable back sheet.

FIG. 11 is a side view of the disposable undergarment shown in FIG. 9 when positioned on a human body and aligned below the wearer's waist by at least about 0.5 inches.

### DETAILED DESCRIPTION OF THE INVENTION

For the purposes of promoting and understanding the principles disclosed herein, references are now made to the embodiments illustrated in the drawings and specific language is used to describe the same. It is nevertheless understood that no limitation of the scope of the invention is thereby intended. Such alterations and further modifications in the illustrated device and such further applications of the principles disclosed as illustrated herein are contemplated as would normally occur to one skilled in the art to which this disclosure relates.

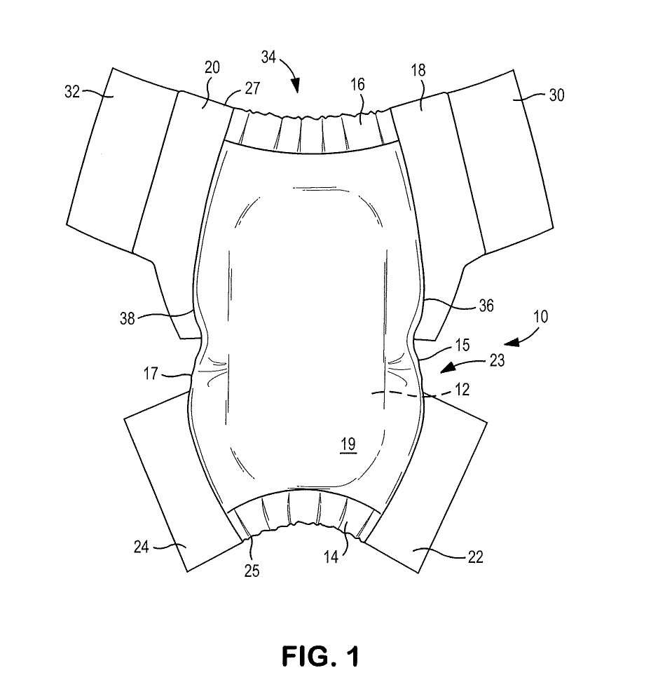

FIGS. 1-4 show one embodiment of the disposable undergarment 10. The disposable undergarment 10 includes an absorbent core 12, a front portion 14, a first side 15, a rear portion 16, a second side 17, first and second extensions 18, 20 respectively, a liquid permeable top sheet 19, a liquid-impermeable back sheet 21, a crotch region 23, first and second tabs 22, 24 respectively, a top edge front portion 25, a first attachment area 26, a top edge back portion 27, a second attachment area 28, first and second flaps 30, 32 respectively, and a main body 34. The disposable undergarment 10 is generally shaped and contoured to be worm close to a wearer's body and below the wearer's waist. By “waist” it is meant the part of a human trunk between the bottom of the rib cage and the pelvis. The wearer's body also includes a buttock and hips. By “buttock” it is meant either of the two rounded prominences of the rear pelvic area. By “hip” it is meant the laterally projecting prominence of the pelvis or pelvic region from the waist to the thigh. When worm, the disposable undergarment 10 is concealed by the wearer's outer garments while being capable of capturing leaks, bodily fluids and waste. The main body 34 is the central portion of the disposable undergarment 10 and includes an absorbent core 12. The absorbent core 12 serves to capture body fluid and body waste and prevents its escape from the disposable undergarment 10. Various structures and layers of materials can be used to construct the main body 34 and the absorbent core 12.

The main body 34 may include a liquid permeable top sheet and one or more liquid-impermeable back sheets. The various layers of material can be formed from paper, plastic or other suitable natural or synthetic fabrics. Such materials can be suitable nonwoven materials. The materials closest to a wearer's body are often made of breathable or permeable materials such that body fluid and/or body wastes are permitted to pass through such materials and are kept away from the wearer's skin. The outer layer can be made from liquid-impermeable materials such that bodily fluid and/or body wastes are not permitted to escape or otherwise leak from the disposable undergarment 10. Elastic bands or other materials can be fixed between the layers of material or to the layers of material to provide elasticity, gathers or other features to conform the disposable undergarment 10 to the body of the wearer. The absorbent core 12 may include one or more acquisition and/or liquid absorption layers that are intended to capture and hold bodily fluids and body waste. Known methods of connecting the various layers of the main body 34 may be used to construct the disposable undergarment 10 such as thermal bonding, adhesive, ultrasonic sealing and various stitching methods.

The main body 34 may also include a front portion 14 and rear portion 16. As can be appreciated, in use, the front portion 14 is configured to fit against the front of a wearer's waist area. The rear portion 16 is configured to fit against the rear of a wearer's waist area, under the wearer's lower back. As stated earlier, the disposable undergarment 10 is a low-rise product such that it can be concealed under a wearer's clothing. Desirably, the disposable undergarment 10 is positioned at least about 1 inch below the wear's waist. More desirably, the disposable undergarment 10 is positioned at least about 1.5 inches below the wear's waist. Even more desirably, the disposable undergarment 10 is positioned at least about 2 inches below the wear's waist. Most desirably, the disposable undergarment 10 is positioned from between about 1 inch to about 3 inches below the wear's waist.

The front portion 14 and rear portion 16 of the disposable undergarment 10 are configured to be positioned on a wearer's body under and away from the waist region of the wearer's clothing. The front portion 14 and the rear portion 16 may include elastic bands, elastic strips or other features to contour the disposable undergarment 10 against the wearer's body. Furthermore, the elasticity provided at the front portion 14 and rear portion 16 can provide the benefit of permitting the disposable undergarment 10 to be worn by wearers of various shapes and sizes while still reliably capturing and retaining bodily fluids and body waste. The elastic bands, strips or other extensible members can be embedded between layers of the main body 34 at the front portion 14 and rear portion 16 or they can be connected at these locations via various methods of attachment, as discussed above, such as thermal bonding, adhesive, ultrasonic sealing or stitching.

Along the longitudinal sides of the main body 34 (i.e., in a direction extending between the front portion 14 and the rear portion 16), various members or panels may be attached to assist with the attachment of the disposable undergarment 10 to the body of a wearer. In the example shown in FIGS. 1-4, the disposable undergarment 10 includes first and second extensions 18, 20, first and second flaps 30, 32, and first and second tabs 22, 24. The first and second extensions 18, 20 are connected to the main body 34 near the rear portion 16 and are further connected to the main body 34 along the longitudinal sides of the main body 34 from rear portion 16 toward the center of the main body 34. The first and second extensions 18, 20 can be separate pieces attached to the main body 34 or they can be extensions of one or more layers of the material that is used to construct the main body 34. The first and second extensions 18, 20 are desirably made of an elastic material, such as an extensible laminate or a breathable partial laminated nonwoven material. Other suitable materials may also be used.

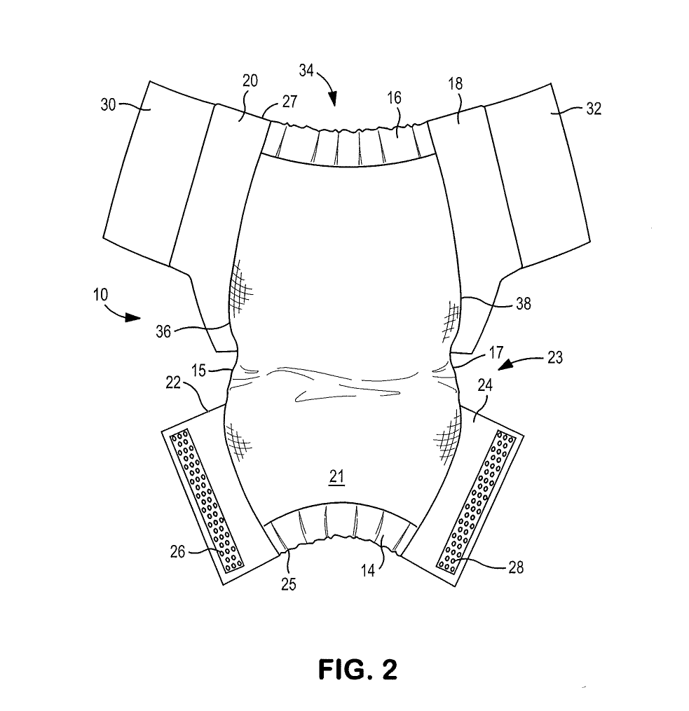

As can be seen in FIGS. 1 and 2, the first and second extensions 18, 20 are connected to the main body 34 at the proximal ends 36 and 38. At distal ends of the first and second extensions 18, 20, the first and second flaps 30, 32 are connected. The first and second flaps 30, 32 are second pieces of material connected to and extending in a transverse direction away from the main body 34. The first and second flaps 30, 32 have a width that is measured in a direction that is generally parallel to a top edge of the rear portion 16 and a height that is measured in a direction that is generally perpendicular to the width. In this example, the height of the first flap 30 is at least approximately twice as long as the width. While various sizes of the disposable undergarment 10 can be constructed, in one configuration, the first and second flaps 30, 32 are approximately 55-65 mm wide and 100-130 mm long. Other sizes and shapes of the first and second flaps 30, 32 can also be used.

The first and second flaps 30, 32 can be connected to the first and second extensions 18, 20 via any suitable means. Suitable methods of attachment may include, but are not limited to, adhesive, stitching, thermal bonding, ultrasonic sealing and the like. In one embodiment, the first and second flaps 30, 32 are secured via an over lock stitch to the first and second extensions 18, 20. The first and second flaps 30, 32 are preferably made of a stretchable loop material. The stretchable loop material is elastic and provides the advantage of contouring the flaps 30, 32 to a wearer's body and accommodating a wide variety of wearer body types and shapes. In one example, the first and second flaps 30, 32 are made of a stretchable loop material that is a woven or knit material that is capable of being extended to at least twice its initial length. In other examples, the stretchable loop material can be extended to 220% of its original, non-extended length. The stretchable loop material additionally has the property that it can securely engage with the hook portion of a typical hook-and-loop fastening system. In this manner, and as will be described, the first and second flaps 30, 32 can directly engage an engaging element of a hook- and-loop fastening system. This property allows the first and second flaps 30, 32 to be made of a single material without the need for manufacturing the flaps 30, 32 from multi-layers of material or to connect a separate fastening mechanism or a separate piece of loop material to the flap 30, 32. This property of the stretchable loop material of the first and second flaps 30, 32 also permits the engaging of the flaps 30, 32 at any position along its entire surface. The stretchable loop material permits such engagement, disengagement and re-engagement a significant number of times.

These properties provide a significant benefit in providing flexibility, adjustment and comfort for a wearer.

The stretchable loop material, with the properties described above, can be constructed of different materials using different types of fabrics and threads. In one example, the stretchable loop material is formed from a stitch bonded fabric that includes a non-bonded fibrous layer in which elastic yarns, such as spandex elastomeric yarns, are stitched to create rows in the fabric with differing abilities to stretch. In this example, the stretchable loop material provides for stretch of 190% in one row and for stretch of 60% in a second row. Substantially perpendicularly from the direction of stretch described above, in this example the stretchable loop material also provides for 80% to 90% stretch in a second direction as well. In this example, the stretchable loop material has a significant overall stretch in the first direction, as well as no more than 90% stretch in a substantially perpendicular direction.

In a desired embodiment of the above example of the stretchable loop material, two types of threads are knitted into the substrate fabric. A first spandex thread that is covered with a texturized nylon can be used in addition to a second polyester thread. The first spandex thread, with a texturized nylon cover, is knitted into the substrate fabric at predetermined intervals in one direction using a simple chain stitch. The second polyester thread can then be knitted into the substrate fabric in one direction using a tricot stitch. These two types of threads can give the stretchable loop fabric a striped or banded appearance.

As discussed above, one of the threads or yarns used in the stretchable loop material can be textured. Textured yarn or thread is subjected to a procedure to increase the volume and the elasticity of the thread or yarn. Textured yarn exhibits twists, crimps, or other distortions along its filaments. In addition to increased elasticity, textured yarns and threads can impart other advantageous properties to fabrics in which it is woven, stitched or otherwise used. One such advantage can be the ability to engage with the hook portion of a hook-and-loop fastening system. The twists, crimps or other distortions give the fabric into which the textured yarn or thread is incorporated a “fuzziness” that can increases its ability to engage and re-engage the hook of a hook-and-loop fastening system.

In one example of the disposable undergarment 10, the first and second flaps 30, 32 are made of a stretchable loop material such as a knitted fabric of the same construction as a bed skirt knitted fabric available from Xymid, LLC of Midlothian, Va. Another example of a stretchable loop material is a polyester knitted fabric sold as a cover for a mattress by Sinomax USA, Inc. of Houston, Tex. These materials are examples of materials that provide flexibility and extendibility and are able to securely engage to the hook portion of a hook-and-loop fastening system.

U.S. Pat. No. 8,021,735 B2 issued Sep. 20, 2011 to Tsiarkezos and entitled: “STITCHBONDED FABRIC WITH A SUBSTRATE HAVING DIVERSE REGIONAL PROPERTIES” is incorporated by reference and made a part hereof. This patent teaches forming a stitch bonded fabric which can be utilized in the disposable undergarment 10.

In another example of a stretchable loop material, the fabric is a knitted fabric using spandex elastic yarn combined with polypropylene. In this example, the stretchable loop fabric may also have one or more layers that can be knitted from the same type of yarn or from different types of yarns, such as from yarns of polyester. Another example of a knitted fabric stretchable loop material is made of polyester, nylon and spandex yarns. Other suitable woven and knit materials can also be used. Such suitable stretchable loop material's capability to engage the hook portion of a hook-and-loop fastening system can be further improved by subjecting the material to heat treatment prior to manufacturing of the disposable undergarment 10. Such heat treatment can include repeated washing and drying of the material on high heat prior to manufacturing of the disposable undergarment 10. Heat treatment of the material can increase the force required to disengage the material from a hook portion of a hook-and-loop fastening system.

As further shown in FIGS. 1-4, the disposable undergarment 10 can also include first and second tabs 22, 24. The first and second tabs 22, 24 are connected to the main body 34 at a location at or near the front portion 14. The first and second tabs 22, 24 may be elongated pieces of material, as shown, and may extend longitudinally along the longitudinal sides of the main body 34 from front portion 14 toward the center of the main body 34. In one example, the length of the first tab 22 can be generally the same size as the height of the first flap 30. The first and second tabs 22, 24 can be separate pieces of material joined to the main body 34 or they can be extended portions of the one or more layers of material that are used to construct the main body 34. In one example, the first and second tabs 22, 24 are pieces of flexible material such as an extensible laminate. Other suitable materials may also be used.

As further shown in FIGS. 1 and 2, the first and second tabs 22, 24 may include first and second attachment areas 26, 28. The first and second attachment areas 26, 28 are portions of the first and second tabs 22, 24 to which the first and second flaps 30, 32 can be connected. In one example, the first and second attachment areas 26, 28 can be hook portions of a typical hook-and-loop fastening system. A piece of a hook portion of a hook-and-loop fastening system can include a base with a plurality of engaging elements extending therefrom. This piece of a hook portion can be connected to the first and second tabs 22, 24 via any suitable attachment method such as, but not limited to, adhesive, thermal bonding, ultrasonic sealing or stitching. In one example, the first and second attachment areas 26, 28 are connected to and extend along a distal edge of the first and second tabs 22, 24. As shown in FIG. 2, the engaging elements of the first and second attachment areas 26, 28 are located on the garment-facing surface of the first and second tabs 22, 24. In this example, the engaging elements are on the surface of the disposable undergarment 10 which faces the clothes of a wearer and away from the body of a wearer. In this manner, the first and second attachment areas 26, 28 are exposed when the disposable undergarment 10 is positioned on a wearer's body and are readily available for a wearer to secure the first and second flaps 30, 32.

In one example, the first and second attachment areas 26, 28 are pieces of hook material from a hook-and-loop fastening system that are approximately 10-15 mm wide by 90-100 mm long. In other examples of the disposable undergarment 10, the first and second tabs 22, 24 may include one or more first and second attachment areas 26, 28. Instead of or in addition to the first and second attachment areas 26, 28, as shown on FIG. 2, the first and second tabs 22, 24 may include a plurality of attachment areas. Several individual pieces of the hook portion of a hook-and-loop fastening system can be connected to the first and second tabs 22, 24. The addition of more than one attachment area can provide the advantage of increased versatility and permit the disposable undergarment 10 to be fitted to a variety of body shapes and sizes. In still another example, additional attachment areas can be placed on the main body 34 at the garment-facing side of the main body 34 to provide additional areas where the first and second flaps 30, 32 can directly attach. The attachment areas can be in a variety of shapes and patterns so as to provide further variations on the fit and positioning of the disposable undergarment 10.

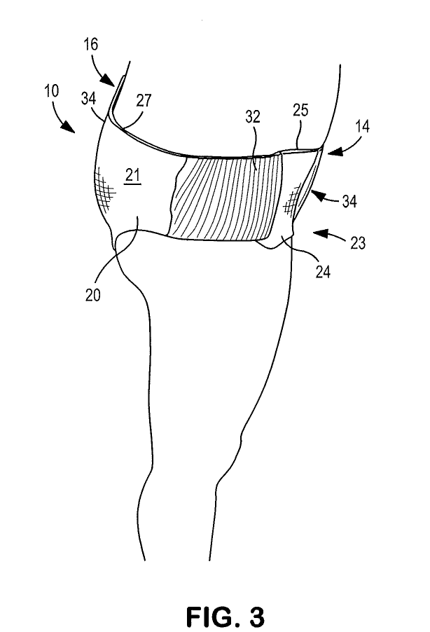

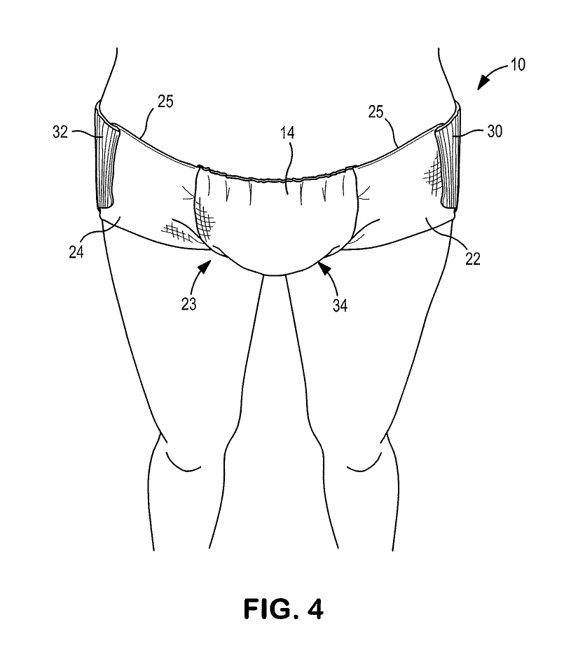

FIGS. 3 and 4 show one embodiment of the disposable undergarment 10 secured to a wearer's body below the wearer's waist. As can be seen, the disposable undergarment 10 is a low-profile or low-rise product, such that it can easily be concealed beneath the wearer's outer clothing. Additionally, it is removable, disposable and capable of fitting a wide variety of wearers including wearers, whose respective weights may differ by 20 pounds or more. When secured on a wearer, the disposable undergarment 10 wraps around the buttock and hip area of a wearer and is positioned below the wearer's waist. In this position, the upper edges of the first and second extensions 18, 20, the first and second flaps 30, 32, and the first and second tabs 22, 24 are coupled with the upper edges of the front portion 14 and the rear portion 16 circumferentially to encompass the crotch region of the wearer. The main body portion 34, the upper edges of front portion 14 and the rear portion 16 occupy a relatively small portion of the circumferential waist region of the disposable undergarment 10. The majority (or more than half) of a wearer's waist is not touched by the main body 34. In other embodiments and sizes of the disposable undergarment 10, the main body 34 can occupy even less of the circumferential waist of the disposable undergarment 10. In one such example, the main body 34 occupies only one-third of a wearer's waist region. The majority of a wearer's waist region is wrapped by the first and second extensions 18, 20, the first and second flaps 30, 32 and the first and second tabs 22, 24. The extensible nature of these elements provides a form-fitting and unobtrusive disposable undergarment 10. This structure of the disposable undergarment 10 also provides the flexibility to secure to wearers of a variety of sizes and shapes.

As previously described, the material of the first and second flaps 30, 32 is configured of stretchable loop material such that it can be directly secure to the first and second attachment areas 26, 28. No separate piece of loop material, which is part of a hook-and-loop fastening system, is required. The material of the first and second flaps 30, 32 can directly be secure to the first and second attachment areas 26, 28 at any location along their surfaces. This also adds to the flexibility, fit and reliability of the disposable undergarment 10. A wearer need only hold the main body 34 and/or the first tab 22 in position and pull or stretch the first flap 30 into position and apply pressure at the first attachment area 26 to secure one side of the disposable undergarment 10 in position. This can be repeated on the second side of the disposable undergarment 10 to fix the disposable undergarment 10 in position. For smaller or thinner wearers, the first and/or second flap 30, 32 can be pulled into an overlapping position for a tighter fit. In such a situation, the distal end of the first and/or second flap 30, 32 would extend over or past the first and second attachment area 26, 28 such that the first and/or second flap 30, 32 is engaged at a location on the first and/or second flap 30, 32 nearer to its connection to the first or second extension 18, 20. The fact that the stretchable loop material can engage the engaging elements at any location on its surface enables the disposable undergarment 10 to be worn by a wider variety of wearers, including wearers with variations in weight of more than twenty pounds.

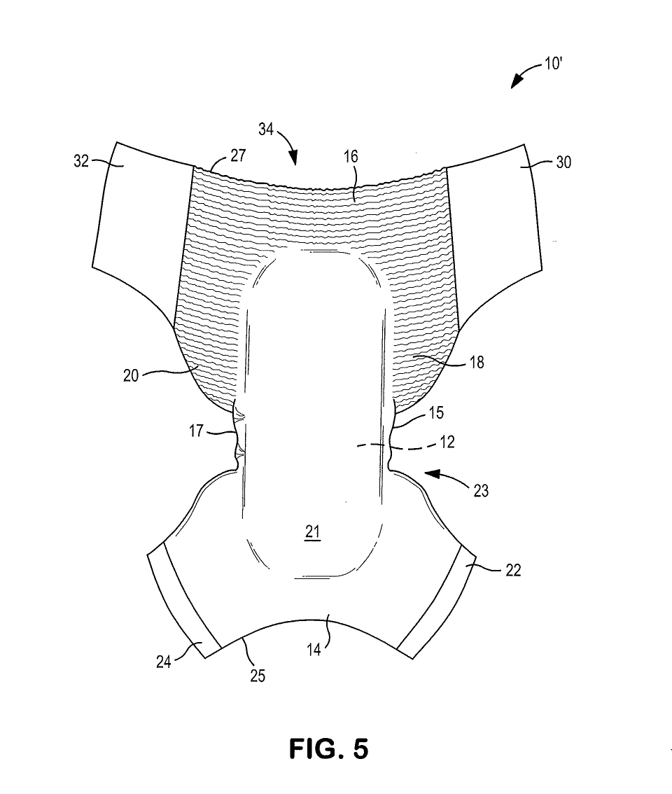

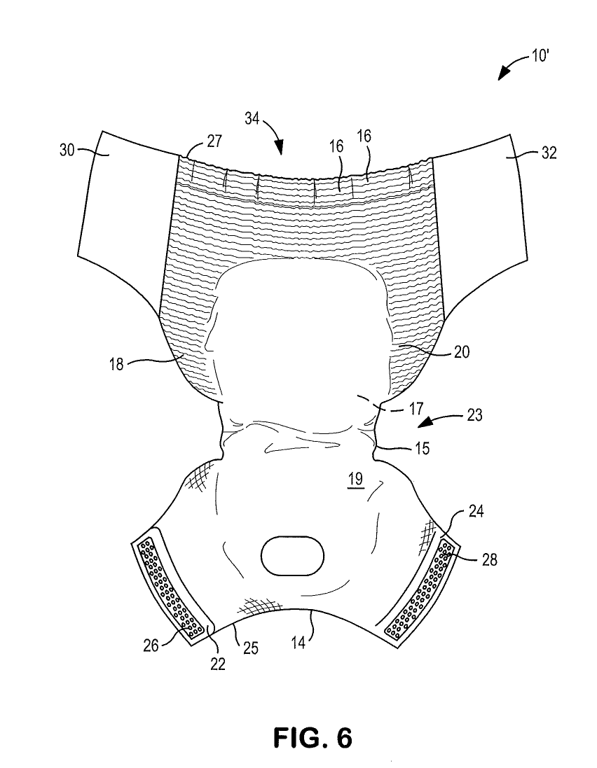

FIGS. 5 and 6 show a second embodiment of the disposable undergarment 10′. As can be appreciated, many of the same elements are included in this embodiment, such as the an absorbent core 12, a front portion 14, a first side 15, a rear portion 16, a second side 17, first and second extensions 18, 20 respectively, a liquid permeable top sheet 19, a liquid-impermeable back sheet 21, a crotch region 23, first and second tabs 22, 24 respectively, a top edge front portion 25, a first attachment area 26, a top edge back portion 27, a second attachment area 28, first and second flaps 30, 32 respectively, and a main body 34. However, as can be seen in this embodiment, the front portion 14 is more curved in shape as are some of the edges of the first and second extensions 18, 20, the first and second flaps 30, 32, and the first and second tabs 22, 24. Various modifications can be made to the relative sizes and shapes of the various elements described herein in an effort to conform the disposable undergarment 10 to various shapes and sizes of wearers.

In still other embodiments of the disposable undergarment 10, the various layers, flaps and attachment components may include other variations of the elements described above and can include variations in which the panels and attachments may be reversed from front to back or from back to front. In another example, the first and second extension 18, 20 and the first and second flap 30, 32 may all be made of a stretchable loop material. The stretchable loop material can also be a continuous piece of material that extends from the main body 34 across the buttocks of a wearer and extend beyond the first and second sides 36, 38 of the main body 34 to create wrap portions that can be releasably attached to the first and second tabs 22, 24. In this example, since the wrap portions are made of stretchable loop material, they can be directly connected to a hook portion of a hook-and-loop fastening system. In other examples, the first and second tabs 22, 24 can also be made of a stretchable loop material. Embodiments of the disposable undergarment 10, wherein the first and second tabs 22, 24 are made of a stretchable loop material, can enable the disposable undergarment 10 to fit a wider range of body sizes, such as wearers with larger waistlines. As explained above, the stretchable loop material may also extend into or over portions of the main body 34 at the front region. Such configurations that use more of the stretchable loop material for portions of the main body 34, as well as for fastening the disposable undergarment 10 around the waist and hips of wearer, can enable a tighter and more body-conforming undergarment.

The disposable undergarment 10 also has an improved design for easier and more sanitary disposal. Once a disposable undergarment 10 captures bodily fluids or other body wastes, it must be disposed. Some existing designs of disposable undergarments permit leakage of bodily fluids or other body wastes, either during use or upon removal and disposal. The disposable undergarment 10 permits a wearer to remove the soiled disposable undergarment 10 from a wearer's body and secure the bodily fluids and waste in the absorbent core 12 for easy and sanitary disposal.

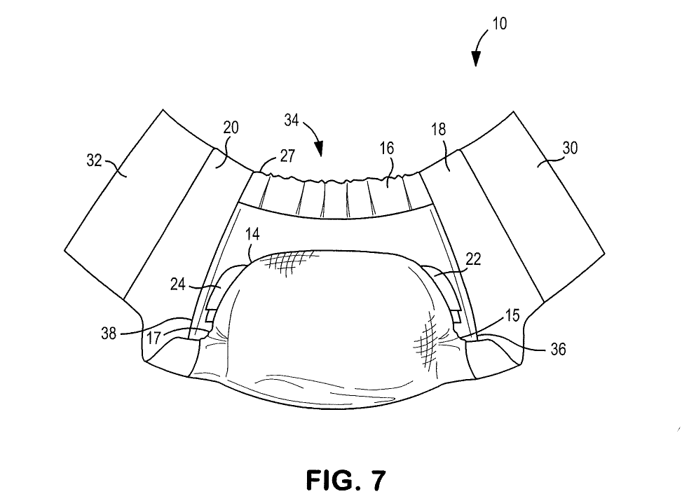

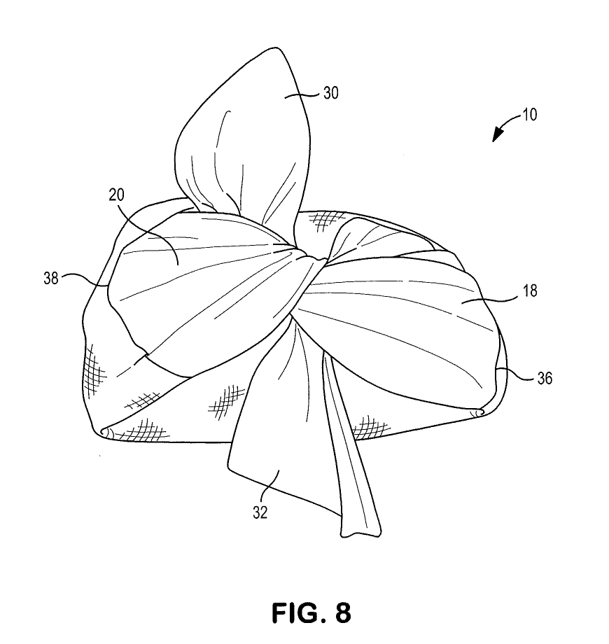

As shown in FIGS. 7 and 8, in order to dispose of the disposable undergarment 10, the front portion 14 can be rolled, folded or otherwise compressed toward the center of the disposable undergarment 10 thus capturing the bodily fluid and/or body waste in the absorbent core 12. As shown in FIG. 7, the front portion 14 is compressed, rolled or folded into a position substantially between the first and second flaps 30, 32. The first and second flaps 30, 32 can then be stretched and tied with an overhand or other suitable knot toward each other. In this manner, the rear portion 16 and the first and second flaps 30, 32 wrap around and encase the front portion and the compressed absorbent core 12 to secure the disposable undergarment 10 in this position for easy and sanitary disposal. The stretchable loop material of the first and second flaps 30, 32 has the elasticity to encompass the compressed the absorbent core 12 and to remain in a tied position as is shown in FIG. 8. As can be appreciated in other alternate embodiments of the disposable undergarment 10, portions of the main body 34, the first and second extension 18, 20, and/or the first and second tab 22, 24 can be made of a stretchable loop material. In these embodiments, additional methods of disposal can also be used to wrap and tie the stretchable loop material around a compressed portion of the disposable undergarment 10.

In still other embodiments of the present disclosure, the disposable undergarment 10 is reusable and not disposable. One or more of the materials can be constructed of natural or synthetic fabrics. The reusable undergarment makes use of the stretchable loop material on the first and second flaps 30, 32 that are attached to the other reusable fabrics that are used to construct the various other portions of the reusable undergarment. In this manner, the low-rise, non-bulky aspects of the disposable undergarment 10, as previously described, can be maintained in a reusable form. The stretchable loop material on the first and second flaps 30, 32 is able to directly engage the hook portion of a hook-and-loop fastening system on or near the front portion of the reusable undergarment.

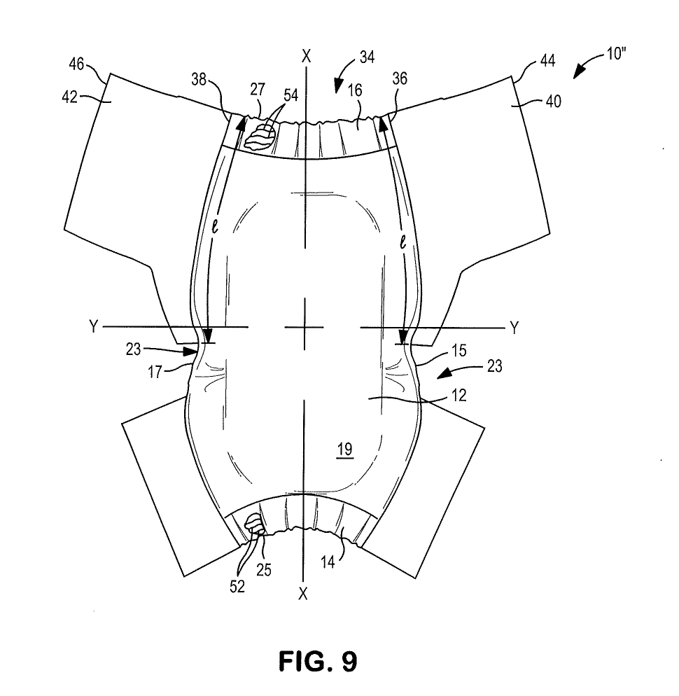

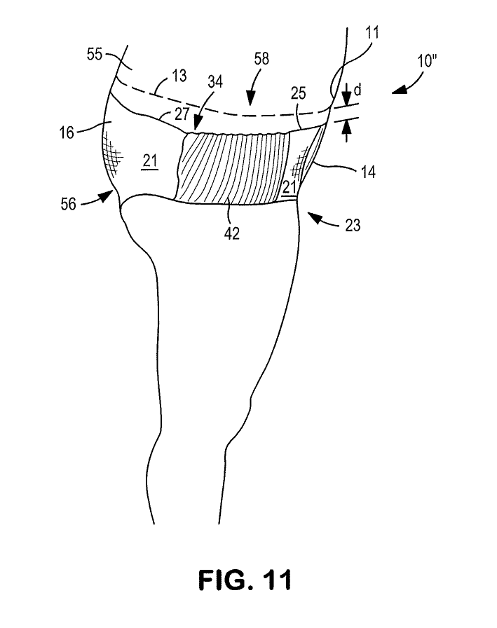

Referring now to FIGS. 9-11, a third embodiment of a disposable undergarment 10″ is depicted. The disposable undergarment 10″ is to be worn on a wearer's body below the wearer's waist 13. By “waist” it is meant the part of a human trunk positioned between the bottom of the rib cage and the pelvis. The waist 13 is depicted in FIG. 11 by a dashed line. The wearer's waist 13 is located a few inches below the wearer's bellybutton 11. By “bellybutton” it is meant the navel.

The disposable undergarment 10″ has a longitudinal central axis X-X and a vertical central axis Y-Y. The disposable undergarment 10″ also has a main body 34 with an absorbent core 12 aligned along the longitudinal central axis X-X. The main body 34 can vary in shape. Desirably, the main body 34 has an hourglass shape. The disposable undergarment 10″ further has a first side 15 and a second side 17, each of which is located laterally outboard of the absorbent core 12. The main body 34 has a liquid permeable top sheet 19, formed from spunbond, which contacts a wearer's body, and a liquid-impermeable back sheet 21, formed from polypropylene, spaced away from the wearer's body. The main body 34 also has a front portion 14, a rear portion 16, and a crotch portion 23. The crotch portion 23 is located between the front and rear portions, 14 and 16 respectively. The front portion 14 has a top edge 25 and the rear portion 16 has a top edge 27. Each of the top edges, 25 and 27, of the front and rear portions, 14 and 16 respectively, is positioned below the wearer's waist 13 when the disposable undergarment 10″ is worn on a wearer's body.

Referring to FIG. 11, each of the top edges 25 and 27 is positioned at a distance (d) below the wearer's waist 13 when the disposable undergarment 10″ is being worn by a human person. The distance (d) is at least about 0.5 inches below the wearer's waist 13 when the disposable undergarment 10″ is being worn. Desirably, the distance (d) is at least about 1 inch below the wearer's waist 13 when the disposable undergarment 10″ is being worn. More desirably, the distance (d) is at least about 1.5 inches below the wearer's waist 13 when the disposable undergarment 10″ is being worn. Even more desirably, the distance (d) is at least about 2 inches below the wearer's waist 13 when the disposable undergarment 10″ is being worm. Most desirably, the distance (d) ranges from between about 0.5 inches to about 3 inches below the wearer's waist 13 when the disposable undergarment 10″ is being worn.

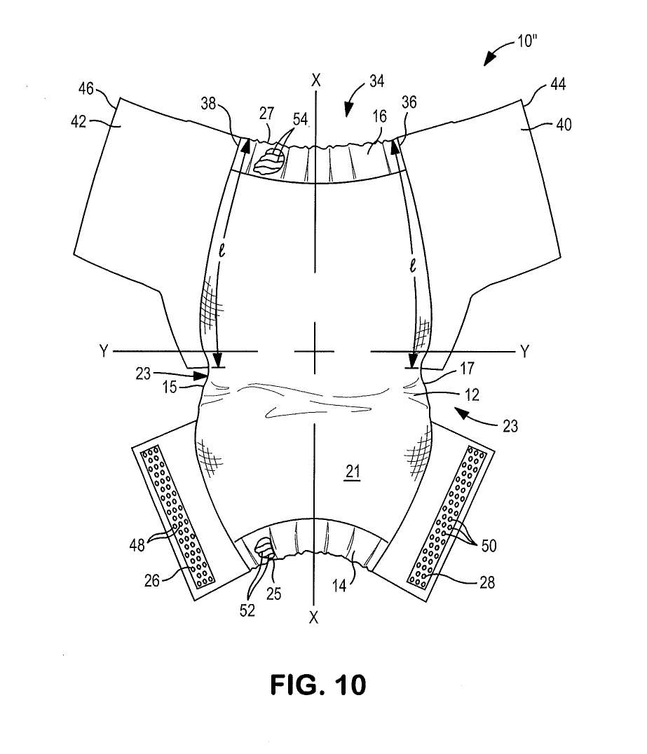

Referring again to FIGS. 9 and 10, the disposable undergarment 10″ also includes a first elastic extension 40 and a second elastic extension 42. Each of the first and second elastic extensions, 40 and 42 respectively, can vary in configuration. For example, each of the first and second elastic extensions, 40 and 42 respectively, can have a quadrilateral shape. By “quadrilateral” it is meant a plane figure with four sides and four angles. In another example, each of the first and second elastic extensions, 40 and 42 respectively, can have a trapezoidal shape. By “trapezoid” it is meant a quadrilateral having two parallel sides. The first and second elastic extensions, 40 and 42 respectively, can have other geometrical shapes, if desired. In addition, the first elastic extension 40 can vary in configuration from the second elastic extension 42. From a manufacturing standpoint, each of the first and second elastic extensions, 40 and 42 respectively, should have the same geometrical shape.

Each of the first and second elastic extensions, 40 and 42 respectively, is constructed of at least about 75% polyester, at least about 3% spandex, and at least about 15% nylon. Desirably, each of the first and second elastic extensions, 40 and 42 respectively, is constructed of at least about 76% polyester, at least about 4% spandex, and at least about 16% nylon. More desirably, each of the first and second elastic extensions, 40 and 42 respectively, is constructed of at least about 78% polyester, at least about 4% spandex, and at least about 16% nylon. Even more desirably, each of the first and second elastic extensions, 40 and 42 respectively, is constructed of at least about 79% polyester, at least about 4% spandex, and at least about 16% nylon. Furthermore, when each of the first and second elastic extensions, 40 and 42 respectively, is constructed of at least about 79% polyester, the polyester includes at least about 50% substrate and at least about 28% yarn.

Each of the first and second elastic extensions, 40 and 42 respectively, can be formed from a uniquely constructed elastic material. Desirably, each of the first and second elastic extensions, 40 and 42 respectively, is formed from a stitch bonded fabric. A stitch bonded fabric can be constructed to have a machine direction, and a cross direction aligned perpendicular to the machine direction. The stitch bonded fabric can have a base layer with a first region, formed from a first substrate material which exhibits a first elongation under a specific tension, and a second region coplanar with the first region, and which exhibits a second elongation under the specific tension that is substantially different from the first elongation. The stitch bonded fabric can exhibit a stitching pattern of threads multi-needle stitched throughout the base layer in rows of stitches running in the machine direction, and having the rows being spaced apart in the cross direction, and in which each of the first and second regions has an area greater than 0.5% of the base layer area. The construction of such a stitch bonded fabric is taught in U.S. Pat. No. 8,021,735 B2 issued on Sep. 20, 2011 to Tsiarkezos. This and other similar stitch bonded fabrics should work well in constructing the disposable undergarment 10″.

Each of the first and second elastic extensions, 40 and 42 respectively, is ultrasonically bonded to both the liquid permeable top sheet 19 and to the liquid-impermeable back sheet 21. The liquid permeable top sheet 19 is formed of spunbond and the liquid-impermeable back sheet 21 is formed of polypropylene. By “ultrasonic bonding” it is meant of or relating to acoustic frequencies above the range audible to the human ear, or above approximately 20,000 hertz. Ultrasonic equipment that can be used to bond the first and second extensions, 40 and 42 respectively, to the first and second sides, 15 and 17 respectively, of the disposable undergarment 10″ is commercially available. One such supplier of such ultrasonic equipment is Sonobond Ultrasonics having an office at 1191 McDermott Drive, West Chester, Pa. 19380. Their Model SM86, equipped with a patterned wheel, model number R1023B, works well. The patterned wheel R1023B can be adjusted to apply a force of at least about 50 pounds per square inch (psi) against the first and second elastic extension material, 40 and 42 respectively, as the material is being ultrasonically bonded to the main body 34. Desirably, the patterned wheel R1023B can be adjusted to apply a force of at least about 55 pounds per square inch (psi) against the first and second elastic extension material, 40 and 42 respectively, as the material is being ultrasonically bonded to the main body 34.

The speed at which the first and second elastic extensions, 40 and 42 respectively, can be ultrasonically bonded to the top sheet 19 and to the back sheet 21 can range from between about 1 inch per second to about 10 inches per second. Desirably, the speed at which the first and second elastic extensions, 40 and 42 respectively, are ultrasonically bonded to the top sheet 19 and to the back sheet 21 is at least about 2 inches per second. More desirably, the speed at which the first and second elastic extensions, 40 and 42 respectively, are ultrasonically bonded to the top sheet 19 and to the back sheet 21 is at least about 2.5 inches per second. Even more desirably, the speed at which the first and second elastic extensions, 40 and 42 respectively, are ultrasonically bonded to the top sheet 19 and to the back sheet 21 is at least about 3 inches per second. Most desirably, the speed at which the first and second elastic extensions, 40 and 42 respectively, are ultrasonically bonded to the top sheet 19 and to the back sheet 21 is greater than about 3 inches per second.

When ultrasonically bonding the first and second elastic extensions, 40 and 42 respectively, the ultrasonic bond should permanently secure the first and second elastic extensions, 40 and 42 respectively, to both the liquid permeable top sheet 19 and to the liquid-impermeable back sheet 21.

It should be noted that this is the first time, to Applicants knowledge, that a polypropylene back sheet 21 was successfully ultrasonically bonded to an elastic material containing at least 75% polyester. Normally, it is very difficult to ultrasonically bond polyester to polypropylene.

Still referring to FIGS. 9 and 10, the first elastic extension 40 has a proximal end 36 and a distal end 44, and the second elastic extension 42 has a proximal end 38 and a distal end 46. The first elastic extension 40 is ultrasonically bonded at the proximal end 36 to the first side 15 of the main body 34. The second elastic extension 42 is ultrasonically bonded at the proximal end 38 to the second side 17 of the main body 34. When ultrasonically bonded, the first elastic extension 40 will extend from the top edge 27 of the rear portion 16 toward the crotch portion 23. Likewise, when ultrasonically bonded, the second elastic extension 42 will extend from the top edge 27 of the rear portion 16 toward the crotch portion 23. The length l of each of the first and second elastic extensions, 40 and 42 respectively, can vary depending on the overall size of the disposable undergarment 10″. Normally, the length l can range from between about 3 inches to about 10 inches. Desirably, the length l can range from between about 4 inches to about 8 inches. More desirably, the length l can range from between about 5 inches to about 7.5 inches.

Referring now to FIG. 10, the disposable undergarment 10″ further includes a first attachment area 26 and a second attachment area 28. Each of the first and second attachment areas, 26 and 28 respectively, has a plurality of engaging elements, 48 and 50 respectively. Each of the first and second attachment areas, 26 and 28 respectively, is secured to the liquid permeable top sheet 19. Each of the first and second attachment areas, 26 and 28 respectively, extends longitudinally from the top edge 25 of the front portion 14 toward the crotch portion 23. The plurality of engaging elements 48, on the first attachment area 26, engage with the first elastic extension 40, and the plurality of engaging elements 50, on the second attachment area 28, engage with the second elastic extension 42 to secure the disposable undergarment 10″ at least about 0.5 inches below the wearer's waist 13. The plurality of engagement elements, 48 and 50, can vary in size, shape and orientation. For example, the plurality of engagement elements, 48 and 50, can be hooks, mushroom shaped hooks, Velcro@ hooks, etc. VELCRO is a registered trademark of Velcro USA having an office at 95 Sundial Avenue, Manchester, N.H. 03103. The plurality of engagement elements, 48 and 50, can include a plurality of hooks. For example, the plurality of engagement elements, 48 and 50, can include from about 10 hooks per square inch to about 2,000 hooks per square inch. More desirably, the plurality of engagement elements, 48 and 50, can include from about 100 hooks per square inch to about 1,500 hooks per square inch. Even more desirably, the plurality of engagement elements, 48 and 50, can include from about 300 hooks per square inch to about 1,000 hooks per square inch. The plurality of engagement elements, 48 and 50, can be randomly arranged or be arranged in a particular pattern.

The plurality of engagement elements, 48 and 50, can be mushroom shaped hooks. The mushroom shaped hooks can be made of plastic or nylon. Desirably, the mushroom shaped hooks are made from 100% plastic. The size, shape, height and dimensions of each mushroom shaped hook can vary as is known to those skilled in the art. One particular mushroom shaped hook that works well in this disposable undergarment 10, 10′ or 10″ is available from Velcro USA, as Model Number HTH 819. Model Number HTH 819 is formed from 100% plastic. The mushroom shaped hooks can be orientated in the machine direction (MD), the cross-direction (CD), or at an angle to the machine direction (MD). The Model Number HTH 819 mushroom shaped hooks perform well for peel and shear when attached to a knit and/or non-woven loop material. The Model Number HTH 819 mushroom shaped hooks provide good peel and exceptional shear. The mushroom shaped hooks can be manufactured in very small configurations which will adequately engage with non-woven materials of all kinds.

Referring again to FIGS. 9 and 10, the front portion 14 of the disposable undergarment 10″ also includes a plurality of elastic bands 52, each of which extends between the first and second sides, 15 and 17 respectively. As depicted, the plurality of elastic bands 52 is sandwiched between the liquid permeable top sheet 19 and the liquid-impermeable back sheet 21. A partial cut away is shown in FIGS. 9 and 10. Each of the plurality of elastic bands 52 is aligned approximately perpendicular to the longitudinal central axis X-X. A portion of the plurality of elastic bands 52, located in the front portion 14, are ultrasonically bonded at the proximal ends 36 and 38, to the first and second elastic extensions, 40 and 42 respectively. The rear portion 16 of the disposable undergarment 10″ also includes a plurality of elastic bands 54, each of which extends between the first and second sides, 15 and 17 respectively. As depicted, the plurality of elastic bands 54 are sandwiched between the liquid permeable top sheet 19 and the liquid-impermeable back sheet 21. A partial cut away is shown in FIGS. 9 and 10. Each of the plurality of elastic bands 54 is aligned approximately perpendicular to the longitudinal central axis X-X. A portion of the plurality of elastic bands 54, located in the rear portion 16, are ultrasonically bonded at the proximal ends 36 and 38, to the first and second elastic extensions, 40 and 42 respectively.

Still referring to FIGS. 9 and 10, each of the first and second elastic extensions, 40 and 42 respectively, is capable of being extended to at least twice its non-extended length in a first direction and no more than about 90% in a second direction. The first direction is aligned perpendicular to the second direction. The plurality of engaging elements, 48 and 50, formed on the first and second attachment areas, 26 and 28 respectively, are hook fasteners. Desirably, the plurality of engaging elements, 48 and 50, formed on the first and second attachment areas, 26 and 28 respectively, are Velcro® hooks. The engagement of the first and second elastic extensions, 40 and 42 respectively, with the first and second attachment areas, 26 and 28 respectively, permit the disposable undergarment 10″ to be worn by a wearer having variations in weight of over 20 pounds.

### Method of Affixing a Disposable Undergarment onto a Wearer's Body

A method of affixing a disposable undergarment 10, 10′ or 10″ around a crotch portion 23 of a wearer's body 55, below their waist 13, is also taught. The wearer's body 55 has a buttock 56 and hips 58. The method includes utilizing a disposable undergarment 10, 10′ or 10″ which includes a main body 34 having a longitudinal central axis X-X, an absorbent core 12 aligned along the longitudinal central axis X-X, and a first side 15 and a second side 17. Each of the first and second sides, 15 and 17 respectively, is located laterally outboard of the absorbent core 12. The main body 34 has a liquid permeable top sheet 19, formed of spunbond, which contacts the wearer's body 55 and a liquid-impermeable back sheet 21, formed from polypropylene, and which is spaced away from the wearer's body 55. The main body 34 also has a front portion 14, a rear portion 16, and a crotch portion 23 located between the front and rear portions, 14 and 16 respectively. Each of the front and rear portions, 14 and 16 respectively, has a top edge, 25 and 27 respectively.

The disposable undergarment 10, 10′ or 10″ also has a first elastic extension 40 and a second elastic extension 42. Each of the first and second elastic extensions, 40 and 42 respectively, is constructed of at least about 75% polyester, at least about 3% spandex, and at least about 15% nylon. Each of the first and second elastic extensions, 40 and 42 respectively, has a proximal end, 36 and 38 respectively, and a distal end, 44 and 46 respectively. The first elastic extension 40 is ultrasonically bonded, at the proximal end 36, to the first side 15 of the main body 34. The second elastic extension 42 is ultrasonically bonded, at the proximal end 38, to the second side 17 of the main body 34. Each of the first and second elastic extensions, 40 and 42 respectively, extends from the top edge 27 of the rear portion 16 toward the crotch portion 23.

The disposable undergarment 10, 10′ or 10″ further has a first attachment area 26 and a second attachment area 28. Each of the first and second attachment areas, 26 and 28 respectively, has a plurality of engaging elements, 48 and 50 respectively. Each of the first and second attachment areas, 26 and 28 respectively, is secured to the liquid permeable top sheet 19 and each extends longitudinally from the top edge 25 of the front portion 14 toward the crotch portion 23. The plurality of engaging elements 48 on the first attachment area 26 engage with the first elastic extension 40 and the plurality of engaging elements 50 on the second attachment area 28 engage with the second elastic extension 42, to secure the disposable undergarment 10, 10′ or 10″ at least about 0.5 inches below the wearer's waist 13.

The method also includes holding the rear portion 16 of the disposable undergarment 10, 10′ or 10″ against the wearer's buttock 56 and below the waist 13. The front portion 14 of the disposable undergarment 10, 10′ or 10″ is then wrapped around the crotch portion 23 of the wearer's body 55. The first elastic extension 40 is stretched around one of the wearer's hips 58 and is secured to the plurality of engaging elements 48 formed on the first attachment area 26. The second elastic extension 42 is then stretched around the wearer's other hip 58 and is secured to the plurality of engaging elements 50 formed on the second attachment area 28, such that the disposable undergarment 10, 10′ or 10″ is secured to the wearer's body 55 below the waist 13. When the disposable undergarment 10, 10′ or 10″ is so secured, the top edge 27 of the rear portion 16 and the top edge 25 of the front portion 14 are positioned at least about 0.5 inches below the wearer's waist 13. Desirably, when the disposable undergarment 10, 10′ or 10″ is so secured, the top edge 27 of the rear portion 16 and the top edge 25 of the front portion 14 are positioned at least about 1 inch below the wearer's waist 13. More desirably, when the disposable undergarment 10, 10′ or 10″ is so secured, the top edge 27 of the rear portion 16 and the top edge 25 of the front portion 14 are positioned at least about 1.5 inches below the wearer's waist 13. Even more desirably, when the disposable undergarment 10, 10′ or 10″ is so secured, the top edge 27 of the rear portion 16 and the top edge 25 of the front portion 14 are positioned from between about 0.5 inches to about 3 inches below the wearer's waist 13.

### Method of Removing a Soiled Disposable Undergarment from a Wearer's Body

A method of removing a soiled disposable undergarment 10, 10′ or 10″ from around a crotch portion 23 of a wearer's body 55 is also taught. The wearer's body 55 has a buttock 56 and hips 58. The method includes utilizing a disposable undergarment 10, 10′ or 10″ which includes a main body 34 having a longitudinal central axis X-X, an absorbent core 12 aligned along the longitudinal central axis X-X, and a first side 15 and a second side 17. Each of the first and second sides, 15 and 17 respectively, is located laterally outboard of the absorbent core 12. The main body 34 has a liquid permeable top sheet 19, formed of spunbond, which contacts the wearer's body 55 and a liquid-impermeable back sheet 21, formed of polypropylene, and which is spaced away from the wearer's body 55. The main body 34 also has a front portion 14, a rear portion 16, and a crotch portion 23 located between the front and rear portions, 14 and 16 respectively. Each of the front and rear portions, 14 and 16 respectively, has a top edge, 25 and 27 respectively.

The disposable undergarment 10, 10′ or 10″ also has a first elastic extension 40 and a second elastic extension 42. Each of the first and second elastic extensions, 40 and 42 respectively, is constructed of at least about 75% polyester, at least about 3% spandex, and at least about 15% nylon. Each of the first and second elastic extensions, 40 and 42 respectively, has a proximal end, 36 and 38 respectively, and a distal end, 44 and 46 respectively. The first elastic extension 40 is ultrasonically bonded, at the proximal end 36, to the first side 15 of the main body 34. The second elastic extension 42 is ultrasonically bonded, at the proximal end 38, to the second side 17 of the main body 34. Each of the first and second elastic extensions, 40 and 42 respectively, extends from the top edge 27 of the rear portion 16 toward the crotch portion 23.

The disposable undergarment 10, 10′ or 10″ further has a first attachment area 26 and a second attachment area 28. Each of the first and second attachment areas, 26 and 28 respectively, has a plurality of engaging elements, 48 and 50 respectively. Each of the first and second attachment areas, 26 and 28 respectively, is secured to the liquid permeable top sheet 19 and each extends longitudinally from the top edge 25 of the front portion 14 toward the crotch portion 23. The plurality of engaging elements 48 on the first attachment area 26 engage with the first elastic extension 40, and the plurality of engaging elements 50 on the second attachment area 28 engage with the second elastic extension 42, to secure the disposable undergarment 10, 10′ or 10″ at least about 0.5 inches below the wearer's waist 13.

The method also includes releasing the first and second elastic extensions, 40 and 42 respectively, from the plurality of engaging elements, 48 and 50 respectively, formed on the first and second attachment areas 26 and 28 respectively. The disposable undergarment 10, 10′ or 10″ is then moved away from the wearer's body 55 whereby the wearer's hips 58 and buttock 56 are no longer contacted by the disposable undergarment 10, 10′ or 10″. The front portion 14 is then rolled or folded inward upon itself towards the rear portion 16 to create an approximately cylindrical configuration with the first and second elastic extensions, 40 and 42 respectively, extending outward therefrom.

The method further includes fastening the first and second elastic extensions, 40 and 42 respectively, together whereby the approximately cylindrical configuration is at least partially encased. The first and second elastic extensions, 40 and 42 respectively, can be fastened together by wrapping the first elastic extension 40 around the second elastic extension 42. Alternatively, the first and second elastic extensions, 40 and 42 respectively, can be fastened or tied together into a knot. Lastly, the method includes disposing of the soiled disposable undergarment 10, 10′ or 10″ in a trash receptacle.

While the invention has been described in conjunction with several specific embodiments, it is to be understood that many alternatives, modifications and variations will be apparent to those skilled in the art in light of the foregoing description. Accordingly, this invention is intended to embrace all such alternatives, modifications and variations which fall within the spirit and scope of the appended claims.

We claim:

1. A disposable undergarment to be worn on a wearer's body below the wearer's waist, comprising:
a) a main body having a longitudinal central axis, an absorbent core aligned along said longitudinal central axis, a first side and a second side, each of said first and second sides being located laterally outboard of said absorbent core, said main body having a liquid permeable top sheet which contacts a wearer's body and a liquid-impermeable back sheet, formed from polypropylene, spaced away from said wearer's body, said main body having a front portion, a rear portion, and a crotch portion located between said front and rear portions, and each of said front and rear portions having a top edge;
b) a first elastic extension and a second elastic extension each constructed of at least about 75% polyester, at least about 3% spandex, and at least about 15% nylon, each of said first and second elastic extensions having a proximal end and a distal end, said first elastic extension ultrasonically bonded at said proximal end to said first side and said second elastic extension ultrasonically bonded at said proximal end to said second side, and each of said first and second elastic extensions extending from said top edge of said rear portion toward said crotch portion; and
c) a first attachment area and a second attachment area each having a plurality of engaging elements, each of said first and second attachment areas secured to said liquid permeable top sheet and each extending longitudinally from said top edge of said front portion toward said crotch portion, and said plurality of engaging elements on said first attachment area engaging with said first elastic extension and said plurality of engaging elements on said second attachment area engaging with said second elastic extension to secure said disposable undergarment at least about 0.5 inches below said wearer's waist.

2. The disposable undergarment of claim 1 wherein said liquid permeable top sheet is formed from spunbond, each of said first and second elastic extensions is a stitch bonded fabric, said stitch bonded fabric is ultrasonically bonded to both said spunbond top sheet and said polypropylene back sheet, and said plurality of engagement elements are mushroom shaped hooks.

3. The disposable undergarment of claim 2 wherein said stitch bonded fabric has a machine direction, and a cross direction aligned perpendicular to said machine direction, said stitch bonded fabric having a base layer with a first region, formed from a first substrate material which exhibits a first elongation under a specific tension, and a second region coplanar with said first region and which exhibits a second elongation under said specific tension that is substantially different from said first elongation, a stitching pattern of threads multi-needle stitched throughout said base layer in rows of stitches running in said machine direction, said rows being spaced apart in said cross direction, in which each of said first and second regions has an area greater than 0.5% of said base layer area.

4. The disposable undergarment of claim 2 wherein each of said first and second elastic extensions has a trapezoidal shape, and said first and second elastic extensions are ultrasonically bonded to both said liquid permeable top sheet and to said liquid-impermeable back sheet using a patterned wheel at an applied force of at least about 55 psi, and at a speed ranging from between about 1 inch per second to about 10 inches per second.

5. The disposable undergarment of claim 1 wherein said top edge of each of said front and rear portions is positioned at least about 1 inch below said wearer's waist when said disposable undergarment is worn on said wearer's body.

6. The disposable undergarment of claim 1 wherein said top edge of each of said front and rear portions is positioned at least about 1.5 inches below said wearer's waist when said disposable undergarment is worn on said wearer's body.

7. The disposable undergarment of claim 1 wherein each of said first and second elastic extensions is constructed of at least about 78% polyester, at least about 4% spandex, and at least about 16% nylon, and said mushroom shaped hooks are formed from 100% plastic.

8. The disposable undergarment of claim 7 wherein said at least about 79% polyester includes at least about 50% substrate and at least about 28% yarn.

9. The disposable undergarment of claim 1 wherein said front portion contains a plurality of elastic bands which extend between said first and second sides, each of said plurality of elastic bands in said front portion is aligned approximately perpendicular to said longitudinal central axis, and a portion of said plurality of elastic bands in said front portion are ultrasonically bonded to said first and second elastic extensions, and said rear portion contains a plurality of elastic bands which extend between said first and second sides, each of said plurality of elastic bands in said rear portion is aligned approximately perpendicular to said longitudinal central axis, and a portion of said plurality of elastic bands in said rear portion are ultrasonically bonded to said first and second elastic extensions.

10. A disposable undergarment to be worn on a wearer's body below their waist, comprising:
a) a main body having a longitudinal central axis, an absorbent core aligned along said longitudinal central axis, a first side and a second side, each of said first and second sides being located laterally outboard of said absorbent core, said main body having an hourglass shape and includes a liquid permeable top sheet which contacts a wearer's body and a liquid-impermeable back sheet, formed from polypropylene, spaced away from said wearer's body, said main body having a front portion, a rear portion, and a crotch portion located between said front and rear portions, and each of said front and rear portions having a top edge;
b) a first elastic extension and a second elastic extension each constructed of at least about 79% polyester, at least about 4% spandex and at least about 16% nylon, each of said first and second elastic extensions having a proximal end and a distal end, said first elastic extension ultrasonically bonded at said proximal end to said first side of said main body and said second elastic extension ultrasonically bonded at said proximal end to said second side of said main body, and each of said first and second elastic extensions extending from said top edge of said rear portion toward said crotch portion; and
d) a first attachment area and a second attachment area each having a plurality of engaging elements, each of said first and second attachment areas secured to said liquid permeable top sheet and each extending longitudinally from said top edge of said front waist portion toward said crotch portion, and said plurality of engaging elements on said first attachment area engaging with said first elastic extension along a line aligned parallel to said longitudinal central axis, and said plurality of engaging elements on said second attachment area engaging with said second elastic extension along a line aligned parallel to said longitudinal central axis to secure said disposable undergarment at least about 0.5 inches below said wearer's waist.

11. The disposable undergarment of claim 10 wherein said liquid permeable top sheet is formed from spunbond, each of said first and second elastic extensions is a stitch bonded fabric, said stitch bonded fabric is ultrasonically bonded to both said spunbond top sheet and said polypropylene back sheet, and said plurality of engagement elements are mushroom shaped hooks formed from 100% plastic.

12. The disposable undergarment of claim 11 wherein said stitch bonded fabric has a machine direction, and a cross direction aligned perpendicular to said machine direction, said stitch bonded fabric having a base layer with a first region formed from a first substrate material which exhibits a first elongation under a specific tension, and a second region which is coplanar with said first region and which exhibits a second elongation under said specific tension that is substantially different from said first elongation, a stitching pattern of threads multi-needle stitched throughout said base layer in rows of stitches running in said machine direction, said rows being spaced apart in said cross direction, and each of said first and second regions has an area greater than 0.5% of said base layer area.

13. The disposable undergarment of claim 10 wherein said top edge of each of said front and rear portions is positioned from between about 0.5 inches to about 3 inches below said wearer's waist when said disposable undergarment is worn on said wearer's body.

14. The disposable undergarment of claim 10 wherein said front portion contains a plurality of elastic bands which extend between said first and second sides, each of said plurality of elastic bands in said front portion is aligned approximately perpendicular to said longitudinal central axis, and a portion of said plurality of elastic bands in said front portion are ultrasonically bonded to said first and second elastic extensions, and said rear portion contains a plurality of elastic bands which extend between said first and second sides, each of said plurality of elastic bands in said rear portion is aligned approximately perpendicular to said longitudinal central axis, and a portion of said plurality of elastic bands in said rear portion are ultrasonically bonded to said first and second elastic extensions.

15. The disposable undergarment of claim 11 wherein each of said first and second elastic extensions is capable of being extended to at least twice its non-extended length in a first direction and no more than about 90% in a second direction, said first direction being aligned perpendicular to said second direction, said plurality of engaging elements formed on said first and second attachment areas are hook fasteners, and engagement of said first and second elastic extensions with said first and second attachment areas permit said disposable undergarment to be worn by a wearer having variations in weight of over 20 pounds.

16. A method of affixing a disposable undergarment around a crotch portion of a wearer's body and below their waist, said wearer's body having a buttock and hips, said method comprising the steps of:
a) utilizing a disposable undergarment which includes a main body having a longitudinal central axis, an absorbent core aligned along said longitudinal central axis, a first side and a second side, each of said first and second sides being located laterally outboard of said absorbent core, said main body having a liquid permeable top sheet which contacts a wearer's body and a liquid-impermeable back sheet, formed from polypropylene, spaced away from said wearer's body, said main body having a front portion, a rear portion, and a crotch portion located between said front and rear portions, and each of said front and rear portions having a top edge; a first elastic extension and a second elastic extension each constructed of at least about 75% polyester, at least about 3% spandex, and at least about 15% nylon, each of said first and second elastic extensions having a proximal end and a distal end, said first elastic extension ultrasonically bonded at said proximal end to said first side of said main body and said second elastic extension ultrasonically bonded at said proximal end to said second side of said main body, and each of said first and second elastic extensions extending from said top edge of said rear portion toward said crotch portion; and a first attachment area and a second attachment area each having a plurality of engaging elements, each of said first and second attachment areas secured to said liquid permeable top sheet and each extending longitudinally from said top edge of said front portion toward said crotch portion, and said plurality of engaging elements on said first attachment area engaging with said first elastic extension and said plurality of engaging elements on said second attachment area engaging with said second elastic extension to secure said disposable undergarment at least about 0.5 inches below said wearer's waist;
b) holding said rear portion of said disposable undergarment against said wearer's buttock and below said waist, and wrapping said front portion around said crotch portion of said wearer's body;
c) stretching said first elastic extension around one of said wearer's hips and securing said first elastic extension to said plurality of engaging elements formed on said first attachment area; and
d) stretching said second elastic extension around said wearer's other hip and securing said second elastic extension to said plurality of engaging elements formed on said second attachment area such that said disposable undergarment is secured to said wearer's body below the waist.

17. The method of claim 16 wherein said top edge of said rear portion and said top edge of said front portion are positioned at least about 1 inch below said wearer's waist when said disposable undergarment is worn on said wearer's body, and said plurality of engagement elements are mushroom shaped hooks formed from 100% plastic.

18. A method of removing a soiled disposable undergarment from around a crotch portion of a wearer's body, said wearer's body having a buttock and hips, said method comprising the steps of:
a) utilizing a disposable undergarment which includes a main body having a longitudinal central axis, an absorbent core aligned along said longitudinal central axis, a first side and a second side, each of said first and second sides being located laterally outboard of said absorbent core, said main body having a liquid permeable top sheet which contacts a wearer's body and a liquid-impermeable back sheet, formed from polypropylene, spaced away from said wearer's body, said main body having a front portion, a rear portion, and a crotch portion located between said front and rear portions, and each of said front and rear portions having a top edge; a first elastic extension and a second elastic extension each constructed of at least about 75% polyester, at least about 3% spandex, and at least about 15% nylon, each of said first and second elastic extensions having a proximal end and a distal end, said first elastic extension ultrasonically bonded at said proximal end to said first side of said main body and said second elastic extension ultrasonically bonded at said proximal end to said second side of said main body, and each of said first and second elastic extensions extending from said top edge of said rear portion toward said crotch portion; and a first attachment area and a second attachment area each having a plurality of engaging elements, each of said first and second attachment areas secured to said liquid permeable top sheet and each extending longitudinally from said top edge of said front portion toward said crotch portion, and said plurality of engaging elements on said first attachment area engaging with said first elastic extension and said plurality of engaging elements on said second attachment area engaging with said second elastic extension to secure said disposable undergarment at least about 0.5 inches below said wearer's waist;
b) releasing said first and second elastic extensions from said plurality of engaging elements formed on said first and second attachment areas;
c) moving said disposable undergarment away from said wearer's body whereby said wearer's hips and buttock are no longer contacted by said disposable undergarment;
d) rolling or folding said front portion inward upon itself towards said rear portion to create an approximately cylindrical configuration with said first and second elastic extensions extending outward therefrom;
e) fastening said first and second elastic extensions together whereby said approximately cylindrical configuration is at least partially encased; and
f) disposing of said soiled disposable undergarment in a trash receptacle.

19. The method of claim 18 further comprising fastening said first and second extensions together by wrapping said first extension around said second extension.

20. The method of claim 18 further comprising fastening said first and second elastic extensions into a knot.
